(19) United States
(12) Patent Application Publication
(10) Pub. No.: US 2019/0008782 A1
(43) Pub. Date: Jan. 10, 2019

(54) POWDER COMPONENT-CONTAINING SEAMLESS CAPSULE AND METHOD FOR MANUFACTURING SAME

(71) Applicant: MORISHITA JINTAN CO., LTD., Osaka (JP)

(72) Inventor: Osami NAKANO, Osaka (JP)

(21) Appl. No.: 16/066,242

(22) PCT Filed: Oct. 21, 2016
(86) PCT No.: PCT/JP2016/081211

§ 371 Date: Jul. 13, 2018

(30) Foreign Application Priority Data
Dec. 28, 2015 (JP) 2015-257062

(51) Int. Cl. A61K 9/48; A61K 35/745; B01J 13/04

(52) U.S. Cl. CPC A61K 9/4816; A61K 35/745; A61K 2035/115; A61K 9/4833; B01J 13/046; A61K 9/4808

(57) ABSTRACT

An object of the present invention is to increase an amount of a powder component dispersed in a content in a seamless capsule. The present invention relates to a seamless capsule including a content and an outer shell enclosing the content adjacently to the content, and provides a powder component-containing seamless capsule in which the content is a suspension prepared by dispersing a powder component, which is poorly soluble in water and oil, in an oil component or a hydrophilic component, the content has a specific gravity of 1 or more, and a difference (Δd=d_{B}−d_{A}) between the specific gravity (d_{A}) of the content and a specific gravity (d_{B}) of the outer shell is within the range of −0.15 to +0.05 and a method for manufacturing the same.

### TECHNICAL FIELD

The present invention relates to a seamless capsule, and relates particularly to a seamless capsule containing a powder component in an amount larger than a conventional one in contents and a method for manufacturing the same.

### BACKGROUND ART

In many cases, seamless capsules can be produced by a dropping method using a multiple nozzle, and oil components are included as a content in the seamless capsules and covered with a water-soluble gel. Among the seamless capsules which have been commercially available, a protective layer which is formed of a hardened oil that protects the content is present between the content and the water-soluble gel.

When bifidobacterium is encapsulated in a seamless capsule, a three-layer structure is generally used in which a suspension prepared by dispersing a powder of bifidobacterium in an oil component is used as a content, and the content is covered with a protective layer formed of a hardened oil component of an outer shell and a water-soluble gel layer formed outside the protective layer. However, when droplets in which a large amount of the bifidobacterium powder is added to the oil component are dropped from a multiple nozzle at the time of manufacture, the droplets may break through the outer protective layer or a liquid layer of the water-soluble gel layer, and thus there has been a limit to the amount of the bifidobacterium powder to be added into the oil component. Taking the bifidobacterium powder as an example, only up to about 20% by weight (a powder component in the whole seamless capsule is 10% by weight) of the bifidobacterium powder has not been able to be added to the contents.

In order to solve the above-mentioned drawbacks in the manufacture of a seamless capsule, Japanese Patent No. 3759986 (Patent Literature 1) proposes that drug crystals are suspended in oil as particles having an average particle size of 20 μm or less, thus obtaining a seamless capsule with an increased amount of drug. In Example 1 of Patent Literature 1, a content liquid contains 15% by weight of β-carotene crystals (content liquid: a suspension prepared by dispersing 75 g of β-carotene crystals in 425 g of medium-chain fatty acid triglyceride (MCT)), and a powder component in the whole seamless capsule is 12% by weight. In Example 2, 40% by weight of nifedipine crystals (content liquid: a suspension prepared by dispersing 200 g of nifedipine crystals in 300 g of MCT) is contained in a seamless capsule. In the case of Example 2, since a film also contains nifedipine, it is as high as 40% by weight; however, when nifedipine is added only into the content liquid without being added into the film, the content of nifedipine in the whole seamless capsule is 20% by weight. However, in the invention of Patent Literature 1, in order to set an average particle size of the drug crystals to 20 μm or less, for example, in the case of β-carotene, following dispersion treatment with a high-speed stirring type homomixer, treatment using a high-pressure homogenizer is required to be carried out three times to adjust the average particle size to 20 μm or less, so that, while the treatment process becomes complicated, operation time tends to be long. In addition, in Example 2 of Patent Literature 1, nifedipine crystals are suspended under a high pressure, so that the treatment process is inevitably complicated.

### CITATIONS LIST

### Patent Literature

Patent Literature 1: Japanese Patent No. 3759986

### SUMMARY OF INVENTION

### Technical Problems

An object of the present invention is to increase an amount of a powder component dispersed in a content of a seamless capsule and to avoid complication of a treatment process or prolongation of treatment time.

### Solutions to Problems

That is, the present invention relates to a seamless capsule including a content and an outer shell enclosing the content adjacently to the content, and provides a powder component-containing seamless capsule in which the content includes a suspension prepared by dispersing a powder component that is poorly soluble in water and oil, in an oil component or a hydrophilic component, the content has a specific gravity of 1 or more, and a difference (Δd=d_{B}−d_{A}) between the specific gravity (d_{A}) of the content and a specific gravity (d_{B}) of the outer shell is within a range of −0.15 to +0.05.

In the powder component-containing seamless capsule of the present invention, preferably, one or more outermost layers enclosing the outer shell on an outer side of the outer shell are provided.

The specific gravity of the outer shell is preferably adjusted by adding a specific gravity regulator.

The specific gravity regulator is preferably selected from the group consisting of inorganic or organic powders having a particle density of 0.9 to 6.0 g/cm³ and a mixture thereof.

The content preferably has a specific gravity of 1.0 to 1.4.

The powder component preferably has an average particle size of more than 20 μm and 150 μm or less.

The powder component is preferably a beneficial enterobacterium.

Preferably, the content includes a suspension prepared by dispersing a bifidobacterium powder in the oil component.

Preferably, the outer shell is a hardened (or hydrogenated) oil whose specific gravity is adjusted to 1.0 to 1.4 by the specific gravity regulator.

Preferably, the outermost layer is a monolayer and is formed from a water-soluble film-forming agent.

The powder component is preferably contained in the content in an amount of 20 to 60% by weight, based on a weight of the content.

The present invention also relates to a three-layer bifidobacterium powder-containing seamless capsule including a content in which a bifidobacterium powder is suspended in an oil component and that has a specific gravity of 1.0 to 1.4, an outer shell that is a hardened oil whose specific gravity is adjusted to 1.0 to 1.4 by a specific gravity regulator and encloses the content adjacently to the content, and an outermost layer that is a monolayer and includes a water-soluble film-forming agent. In this seamless capsule, a difference (Δd=d_{B}−d_{A}) between the specific gravity (d_{A}) of the content and the specific gravity (d_{B}) of the outer shell is within the range of −0.15 to +0.05.

The present invention also relates to a method for manufacturing a seamless capsule, and the method includes simultaneously extruding a content liquid from an inner nozzle of a double nozzle having a sequentially increasing radius and disposed concentrically and an outer shell liquid from an outer nozzle into a cooling liquid. The invention provides a method for manufacturing the foregoing powder component-containing seamless capsule in which the content liquid includes a suspension prepared by dispersing a powder component that is poorly soluble in water and oil, in an oil component or a hydrophilic component, the content liquid has a specific gravity of 1 or more, and a difference (Δd=d_{B}−d_{A}) between a specific gravity (d_{A}) of the content liquid and a specific gravity (d_{B}) of the outer shell liquid is controlled within the range of −0.15 to +0.05.

In the method for manufacturing a powder component-containing seamless capsule, one or more outermost layer nozzles having a sequentially increasing radius and arranged concentrically are further provided outside the double nozzle, and it is conceivable that one or more outermost layer liquids are extruded from the one or more outermost layer nozzles into the cooling liquid simultaneously with the content liquid and the outer shell liquid.

The present invention also relates to a method for manufacturing a three-layer seamless capsule, and the method includes simultaneously extruding a content liquid from an inner nozzle of a triple nozzle having a sequentially increasing radius and including the inner nozzle, an intermediate nozzle, and an outer nozzle arranged concentrically, an outer shell liquid from the intermediate nozzle, and an outermost layer liquid from the outer nozzle into a cooling liquid. The invention provides a method for manufacturing the foregoing three-layer bifidobacterium powder-containing seamless capsule in which the content liquid is a suspension prepared by dispersing a bifidobacterium powder in the oil component and has a specific gravity of 1.0 to 1.4, the outer shell liquid is a hardened oil whose specific gravity is adjusted to 1.0 to 1.4 by the specific gravity regulator, the outermost layer liquid contains the water-soluble film-forming agent, and a difference (Δd=d_{B}−d_{A}) between the specific gravity (d_{A}) of the content liquid and a specific gravity (d_{B}) of the outer shell is controlled within the range of −0.15 to +0.05.

### Advantageous Effects of Invention

The present invention has made it possible to control the specific gravity of a content and the specific gravity of a layer (outer shell) adjacent to the content and increase an amount of a powder component introducing into the content. The present inventor has considered that what the content breaks through an outer shell layer around the content, when droplets are dropped from a multiple nozzle at the time of manufacture of a seamless capsule, is caused by a difference in specific gravity between the content and the outer shell. That is, as shown in FIG. 1, when the specific gravity of the content is higher than the specific gravity of the outer shell, the content tends to break through the outer shell layer downwardly from the lower side of the outer shell due to its weight. On the contrary, when the specific gravity of the content is considerably lower than the specific gravity of the outer shell, as shown in FIG. 2, the content becomes slower than the outer shell and breaks through the outer shell layer upwardly. Accordingly, the present inventor has found that when a difference (Δd=d_{B}−d_{A}) between the specific gravity (d_{A}) of the content and the specific gravity (d_{B}) of the outer shell is within the range of −0.15 to +0.05 if the specific gravity of the content is 1 or more, the content does not break through the outer shell layer and is kept enclosed in the outer shell.

In the present invention, even if a powder component is not ground to an average particle size of 20 μm or less in the content, as long as the specific gravity is adjusted, the powder component can be added into the content in an amount larger than in the prior art. The fact that a large amount of the powder component can be added into the content means that many powder components are contained in a seamless capsule, the size of the seamless capsule can be reduced, and the number of capsules to be taken can be reduced. As a result, the amount of the starting material of the seamless capsule can be reduced, which is useful for resource saving.

### BRIEF DESCRIPTION OF DRAWINGS

FIG. 1 is a schematic diagram of a capsule defect occurring at the time of manufacture of a seamless capsule when the specific gravity of a content is higher than the specific gravity of an outer shell.

FIG. 2 is a schematic diagram of a capsule defect occurring at the time of manufacture of the seamless capsule when the specific gravity of the content is lower than the specific gravity of the outer shell.

FIG. 3 is a schematic cross-sectional view of a nozzle portion of a manufacturing apparatus suitable for manufacturing a three-layer seamless capsule by a dropping method using a triple nozzle.

FIG. 4 is an enlarged photograph of a seamless capsule obtained in Example 1.

FIG. 5 is an enlarged photograph of a seamless capsule obtained in Example 2.

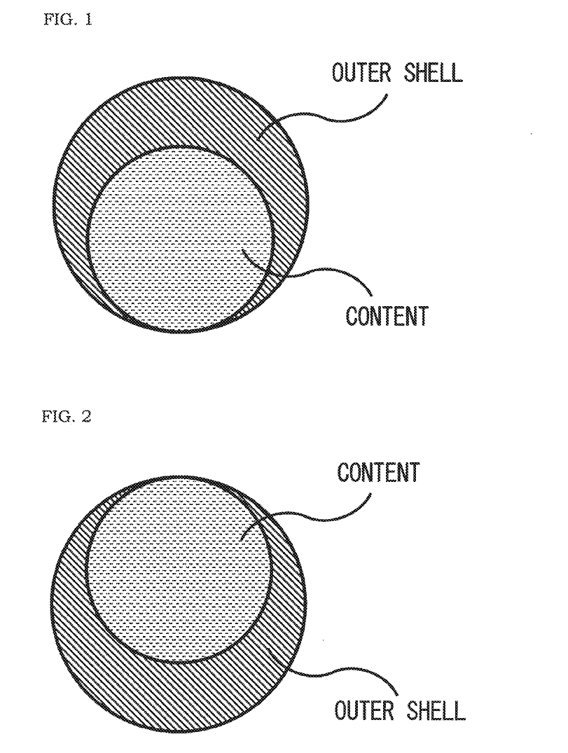

### DEFINITION OF TERMS

The “suspension” as used herein refers to a solution prepared by dispersing a powder component, which is poorly soluble in water and oil, in a liquid such as water, oil, alcohol or a mixture thereof. It is not necessary that the powder component is completely insoluble in the liquid, and the powder component may be partially dissolved; however, the “suspension” means a state in which the undissolved powder component is dispersed in the liquid.

The “specific gravity” as used herein means a specific gravity at 50° C. unless otherwise specified. In addition, the specific gravity means a specific gravity of a starting material prepared solution of each layer in a seamless capsule manufacturing process, unless otherwise specified.

In the present specification, the “average particle size” of the powder component means a particle size at an integrated value of 50% in particle size distribution measured by a laser diffraction/scattering method.

### DESCRIPTION OF EMBODIMENT

### Seamless Capsule

The seamless capsule of the present application basically includes two layers, that is, consisting of a content and an outer shell, but one or more outermost layers may be further provided outside the outer shell. In the present invention, the powder component is dispersed in the content of the seamless capsule. In addition, it is necessary that the specific gravity of the content is 1 or more and a difference (Δd=d_{B}−d_{A}) between the specific gravity (d_{A}) of the content and the specific gravity (d_{B}) of the outer shell is within the range of −0.15 to +0.05.

In the present invention, for the sake of simplicity, a three-layer seamless capsule consisting of a content, an outer shell, and an outermost layer will be described.

### Content

In the seamless capsule of the present invention, the content is a suspension prepared by dispersing a powder component in an oil component or a hydrophilic component. The oil component can be an animal oil, a vegetable oil, a mineral oil, a silicone oil or the like and is not particularly limited; and it may be either liquid or solid at ordinary temperature and can be its hardened oil. Specific examples of suitable oil components include coconut oil composed largely of medium chain fatty acid and a hardened (or hydrogenated) oil thereof, vegetable oil such as sesame oil, coffee oil, rapeseed oil, olive oil, and sunflower oil mainly composed largely of long chain fatty acid and a hardened oil thereof, liquid paraffin, silicone oil and a mixture thereof. It is also possible to add beeswax to the oil components for viscosity adjustment. The hardened oil may be purified one. Particularly preferred examples of the oil component include hardened palm oil (melting point 35° C. to 50° C.: WITOCAN-H and WITOCAN-42/44: both manufactured by Cremer Oleo GmbH & Co. Kg) which is edible hardened oil.

The hydrophilic component is water or alcohols. Specific examples of alcohols include, but not limited to, ethanol, methanol, polyethylene glycol, propylene glycol, glycerin, and a mixture thereof.

The powder component dispersed in the content is not particularly limited, but can preferably be a pharmaceutical ingredient or a functional component, in particular a substance which is weak against acid, moisture, or heat and is solid at ordinary temperature. Specific examples of the powder component include poorly soluble drugs (e.g., acetaminophen), fungi (e.g., beneficial enterobacterium such as bifidobacterium or lactic acid bacteria and yeast), proteins (e.g., lactoferrin), enzymes (e.g., nattokinase), and a mixture thereof. The average particle size of the powder component of the present invention is not limited, but those having a large average particle size, specifically, those having an average particle size of more than 20 μm can be used. In the present invention, even a powder component having an average particle size of more than 20 μm can be dispersed in the content of the seamless capsule. Even if the average particle size of the powder component of the present invention is too large, it is not preferred because it cannot be called to be powder, and the upper limit of the average particle size is 150 μm or less, preferably 120 μm or less, more preferably 100 μm or less.

The amount of the powder component is preferably 20 to 60% by weight, more preferably 25 to 55% by weight, most preferably 40 to 50% by weight, based on the weight of the content. In the present invention, the purpose is to make the amount of the powder component larger than the conventional 20% by weight. However, it is difficult that the powder component can be contained in the seamless capsule in an amount of more than 60% by weight. The preferred content of the powder component in the whole seamless capsules can preferably be 25 to 40% by weight, more preferably 28 to 38% by weight, most preferably 30 to 35% by weight.

In the present invention, it is necessary to disperse the above content in an oil component or a hydrophilic component. Although dispersion can be carried out using an ordinary dispersing machine or a suspension machine, it is unnecessary in the present invention to grind the powder component to an average particle size of 20 μm or less. Thus, it is possible to omit a grinding process such as a grinding process at 10,000 RPM for 5 minutes or more using a high-speed stirring type emulsification/dispersion machine, or three-times grinding process at 100 MPa using a high pressure homogenizer.

In the present invention, the specific gravity of the content is required to be 1.0 or more. When the specific gravity of the content is less than 1.0, it corresponds to a seamless capsule covering a conventional powder component (for example, bifidobacterium powder), and the seamless capsule can contain merely up to about 20% by weight of the weight of the content at the maximum. When the specific gravity of the content is 1.0 or more, in conventional cases the content of the seamless capsule easily pierces through the outer shell. The specific gravity of the content is generally 1.4 or less, preferably 1.2 or less. In the conventional technique, it is difficult to form a seamless capsule whose content has the specific gravity of more than 1.4.

When the seamless capsule of the present invention has a three-layer structure, an outer shell adjacent to the content plays the role of a protective layer for the content, and an outermost layer is further formed outside the outer shell.

### Outer Shell

In the seamless capsule of the present invention, the outer shell is used for the purpose of protecting the content and can be a substance which is less likely to mix with water, suitably including an oleophilic viscous substance and a hardened oil. The oleophilic viscous substance preferably has a viscosity equal to or higher than that of the oil component used for the content. In other words, the oleophilic viscous substances used for the outer shell preferably has a viscosity equal to or higher than that of the oil component used for the content. The viscosity of the oleophilic viscous substance may be adjusted by adding an oil component exemplified by the content as necessary. Examples of the oleophilic viscous substance include sucrose isobutyrate acetate (SAIB) having a specific gravity of 1.146 (25° C.) and tocopherol having a specific gravity of 0.947 to 0.955 (20° C.).

The hardened oil used for the outer shell is only required to protect the content, and can preferably be a hardened oil which secures fluidity inside a nozzle at the time of manufacture and becomes solid at a temperature around ordinary temperature. The hardened oil which exhibits fluidity at a temperature at the time of manufacture and becomes solid at the temperature around ordinary temperature may be the same hardened oil as the hardened oil used for the content. After encapsulation, the solid hardened oil layer can protect the content.

In the present invention, the specific gravity difference (Δd=d_{B}−d_{A}) between the specific gravity (d_{A}) of the content and the specific gravity (d_{B}) of the outer shell is required to be adjusted within the range of −0.15 to +0.05. Accordingly, the specific gravity of the outer shell is adjusted and controlled by adding a specific gravity regulator to the outer shell. In the specific gravity of the outer shell, when the difference (Δd=d_{B}−d_{A}) between the specific gravity (d_{A}) of the content and the specific gravity (d_{B}) of the outer shell is within the range of −0.15 to +0.05 without adding the specific gravity regulator, it is not necessary to add the specific gravity regulator. The difference (Δd=d_{B}−d_{A}) between the specific gravity (d_{A}) of the content and the specific gravity (d_{B}) of the outer shell can preferably be in the range of −0.10 to +0.02, more preferably in the range of −0.05 to +0.01. When the difference (Δd=d_{B}−d_{A}) between the specific gravity (d_{A}) of the content and the specific gravity (d_{B}) of the outer shell falls outside the above range, a phenomenon occurs in which the content goes out of the outer shell, so that seamless capsules are not properly formed.

Examples of the specific gravity regulator include inorganic powder (such as titanium dioxide, calcium carbonate, magnesium carbonate, magnesium oxide, calcium stearate, magnesium stearate, barium sulfate, silicon dioxide, talc, activated carbon, bentonite, and kaolin), organic powder (such as starch, dextrin, corn starch, monosaccharides, disaccharides, and polysaccharides such as trisaccharides or more), and a mixture thereof.

The specific gravity regulator is a powder which is added to an outer shell base material in order to increase the specific gravity of the outer shell at the time when the specific gravity of the content increases and becomes imbalanced with the outer shell by adding a large amount of powder components to the content, The specific gravity regulators can preferably have a particle density within the range of 0.9 to 6.0 g/cm³, more preferably 1.2 to 5.0 g/cm³, most preferably 2 to 4.5 g/cm³. If the specific gravity regulator has a particle density of less than 0.9 g/cm³, a large amount of the specific gravity regulator must be added, and the performance of the outer shell may be deteriorated. If the specific gravity regulator has a particle density of more than 6.0 g/cm³, a difference from the specific gravity of the outer shell becomes large, and there is a possibility that the content goes out of the outer shell when preparing the seamless capsule.

### Outermost Layer

The outermost layer of the present invention contains at least a film-forming agent. The file-forming agent can either be a natural polymer or a synthetic polymer. The natural polymers are generally water-soluble, and examples thereof include, but not limited to, gelatin, casein, zein, pectin and derivatives thereof, alginic acid and its salts, agar, gellan gum, tragacanth gum, guar gum, locust bean gum, carrageenan, furcellaran, tamarind, mannan, hemilose, chitosan, curdlan and the like. These natural polymers may be used solely or in combination of two or more kinds.

The synthetic polymer can be obtained by using a mixture of a photopolymerizable monomer and a photopolymerization initiator as a film-forming agent and polymerizing the film-forming agent by light irradiation. Examples of photopolymerizable monomers that can be used include, but not limited to, hydrophilic monomers such as nona(ethylene glycol) di(meth)acrylate, tetradeca(ethylene glycol) di(meth)acrylate, trimethylolpropane tri(meth)acrylate, ditrimethylolpropane tetraacrylate, pentaerythritol tetraacrylate, bisphenol A di(meth)acrylate; and hydrophobic monomers such as 1,6-hexanediol di(meth)acrylate, 1,9-nonanediol di(meth)acrylate, dimethylol-tricyclodecane di(meth)acrylate, neopentyl glycol diacrylate, and ethylene oxide modified bisphenol A di(meth)acrylate. The photopolymerization initiator is a compound which can generate a polymerization initiation species by light irradiation and promote polymerization reaction or crosslinking reaction. Examples of the photopolymerization initiator include benzoin, acetoin, benzoin methyl ether, benzoin ethyl ether, benzoin isopropyl ether, benzoin isobutyl ether, benzophenone, benzyl Michler's ketone, xanthone, chlorothioxanthone, isopropylthioxanthone, benzyl dimethyl ketal, naphthol, anthraquinone, hydroxyanthracene, acetophenone diethyl ketal, α-hydroxycyclohexyl phenyl ketone, 2-hydroxy-2-methylphenylpropane, an aromatic iodonium salt, an aromatic sulfonium salt, an iodonium salt, a sulfonium salt, a triarylsulfonium salt, a trifluorocarbon sulfonium salt and the like. The polymerization initiator may be used solely or in combination of two or more kinds. Although the synthetic polymer may be crosslinked or noncrosslinked, the above-mentioned photopolymerizable monomer has a plurality of polymerizable groups, so that a crosslinked film is formed.

In addition to the film-forming agent, examples of the outermost layer may contain other components such as plasticizers (specific examples thereof include sorbitol, glycerin, propylene glycol, and polyethylene glycol), bulking agents (specific examples thereof include dextrin and starch), coloring agents, flavoring agents, sweetening agents, acidulants and the like. These components may be used solely or in combination of two or more kinds.

### Method for Manufacturing Seamless Capsule

The dispersed or suspended content is encapsulated by the outer shell and, if necessary, by the outermost layer; but the method of encapsulation is not particularly limited. The most preferable encapsulation method can be a method, generally called a dropping method, wherein the content is dropped into a coagulation liquid using a double, triple, or more concentric nozzle (for example, Japanese Unexamined Patent Application Publication Nos. 849-59789, S51-8176, and S60-172343). When a photocurable synthetic polymer is used as a film-forming agent in the outermost layer, the methods described in Japanese Unexamined Patent Application Publication No. 2009-278874 and WO2012/060417 can be applied. In particular, the present invention can be applied to seamless capsules having three or more layers, for example, the multiple capsule described in Japanese Unexamined Patent Application Publication No. H11-079964. As described above, in order to manufacture the three-layer seamless capsule having the content, the outer shell, and the outermost layer that is a monolayer, a description will be given using a triple nozzle.

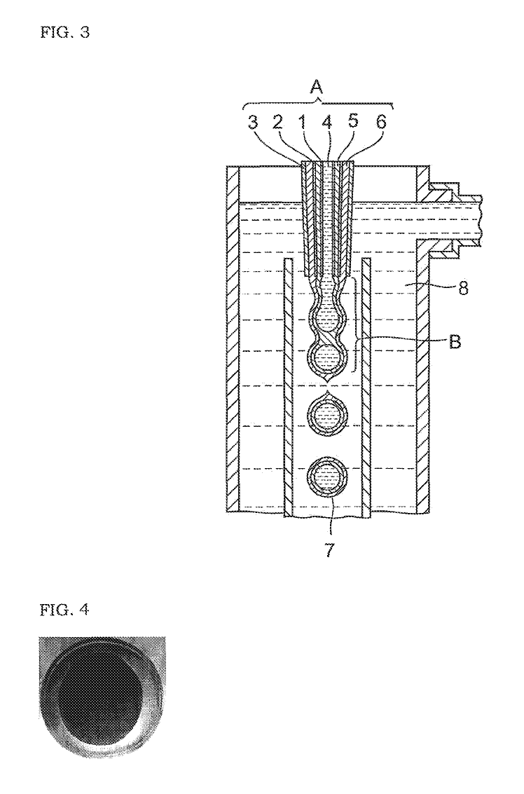

FIG. 3 shows a schematic cross-sectional view of a nozzle portion of a manufacturing apparatus suitable for manufacturing a three-layer seamless capsule by a dropping method using a triple nozzle.

FIG. 3 shows a state in which a seamless capsule jet B ejected from a nozzle section A is cut in a coolant 8 to become each seamless capsule 7. In the nozzle section A, an inner nozzle 1, an intermediate nozzle 2, and an outer nozzle 3 are present concentrically. A capsule content liquid 4 is ejected into the inner nozzle 1, an outer shell liquid 5 is ejected from the intermediate nozzle 2 (specifically, between the intermediate nozzle 2 and the inner nozzle 1), and an outermost layer liquid 6 is simultaneously ejected from the outer nozzle 3 (specifically, between the outer nozzle 3 and the intermediate nozzle 2).

The seamless capsule obtained as described above may be air-dried at 5 to 30° C. for 2 to 12 hours. Vacuum drying or vacuum freeze drying may be carried out after air drying. In the vacuum drying, a degree of vacuum is kept at a range of 0.0002 to 0.5 MPa, and in the vacuum freeze drying, freeze-drying is performed at −20° C. or less. The time required for vacuum drying or vacuum freeze drying is not particularly limited, but is generally 5 to 60 hours, preferably 24 to 48 hours. If the time is 5 hours or less, the drying becomes insufficient and, therefore, the content may have a bad influence from water present in the capsule.

The size of the seamless capsule of the present invention is not particularly limited, but it is desirable that the seamless capsule has a diameter of 0.5 to 10 mm, preferably 1 to 8 mm. If the diameter of the seamless capsule is less than 0.5 mm, the amount of the content of the seamless capsule decreases and many capsules are necessary. Even if the diameter is more than 10 mm, it does not cause any problem, but preferably, the diameter is not more than 10 mm because of the ease of handling.

Although the case where the outermost layer of the foregoing seamless capsule is a monolayer is described, two or more outermost layers may be provided. When two or more outermost layers are provided, a layer having a specific function may be provided in addition to a film layer, and, for example, an acid resistant layer resistant to acid in the stomach may be considered. The layers having a specific function may be formed on either the outer or inner side of the film layer.

In a particularly preferred embodiment of the present invention, the seamless capsule is a three-layer bifidobacterium powder-containing seamless capsule. The three-layer bifidobacterium powder-containing seamless capsule is manufactured by a method for manufacturing a three-layer seamless capsule, and the method includes simultaneously extruding a content liquid from an inner nozzle of a triple nozzle having a sequentially increasing radius and including the inner nozzle, an intermediate nozzle, and an outer nozzle arranged concentrically, an outer shell liquid from the intermediate nozzle, and an outermost layer liquid from the outer nozzle into a cooling liquid. In this method, the content liquid is a suspension prepared by dispersing a bifidobacterium powder in an oil component and has a specific gravity of 1.0 to 1.4, the outer shell liquid is a hardened oil whose specific gravity is adjusted to 1.0 to 1.4 by a specific gravity regulator, the outermost layer liquid contains a water-soluble film-forming agent, and a difference (Δd=d_{B}−d_{A}) between a specific gravity (d_{A}) of the content liquid and a specific gravity (d) of the outer shell is controlled within the range of −0.15 to +0.05, whereby the three-layer bifidobacterium powder-containing seamless capsule can be manufactured.

### Two-Layer Seamless Capsule

Although the foregoing seamless capsule is based on the case of three or more layers, a two-layer seamless capsule having only the content and the outer shell is also within the scope of the present invention. In the case of the two-layer seamless capsule, the outer shell adjacent to the content is often a water-soluble film-forming agent used for the outermost layer. In the case of the two-layer seamless capsule, for the specific gravity of the outer shell which is the film-forming agent layer, the difference (Δd=d_{B}−d_{A}) between the specific gravity (d_{A}) of the content and the specific gravity (ds) of the outer shell is required to be adjusted within the range of −0.15 to +0.05, and there is also a case where the specific gravity regulator is mixed with the water-soluble film-forming agent in order to adjust the specific gravity. A two-layer seamless capsule is manufactured by using a double nozzle instead of a triple nozzle in the same manner as in the case of the triple nozzle.

The two-layer seamless capsule of the present invention is manufactured by a method for manufacturing a seamless capsule, and the method includes simultaneously extruding a content liquid from an inner nozzle of a double nozzle having a sequentially increasing radius and disposed concentrically and an outer shell liquid from an outer nozzle into a cooling liquid. In this method, the content liquid includes a suspension prepared by dispersing a powder component, which is poorly soluble in water and oil, in an oil component or a hydrophilic component, the content liquid has a specific gravity of 1 or more, and the difference (Δd=d_{B}−d_{A}) between the specific gravity (d_{A}) of the content liquid and the specific gravity (d_{B}) of the outer shell liquid is controlled within the range of −0.15 to +0.05, whereby the two-layer seamless capsule can be manufactured.

### EXAMPLES

The present invention will be explained in more detail by examples. The present invention should not be construed as being limited to the examples.

### Reference Example

In this reference example, a three-layer bifidobacterium seamless capsule that is currently actually prescribed is exemplified, and a maximum amount of bifidobacterium is added. In this reference example, a seamless capsule was formed without adding a specific gravity regulator in the outer shell prescription.

(a) Commercially available bacteria powder (the number of organisms being 1.5×10¹¹ number/g and the average particle size being 105 μm) obtained by freeze-drying a mixture of bifidobacterium longum organisms with a protective agent was dispersed in a hardened oil dissolved at a melting point of 37° C. such that 20% by weight was achieved, and the resulting solution was used as a content liquid. The specific gravity (d_{A}) of the content liquid was 0.978.

(b) Hardened coconut oil (product name: WITOCAN-42/44) having a melting point of 43° C. was used as an outer shell liquid. The specific gravity (d_{B}) of the hardened coconut oil was 0.904.

(c) 18.5% by weight of gelatin, 6% by weight of a plasticizer (glycerin) and 0.5% by weight of thickening polysaccharide (pectin) were dissolved in 75% by weight of purified water to adjust the solid content to 25%, and thus to prepare a film solution.

A triple coaxial nozzle as shown in FIG. 3 was prepared, in which the content liquid (a) was put in an inner nozzle, the outer shell liquid (b) was put in an intermediate nozzle outside the inner nozzle, and the film liquid (c) was put in the outer nozzle, that is, the three liquids (a) to (c) were simultaneously dropped into an oil which was cooled and flowed, to form seamless capsules having a three-layer structure, which have a diameter of 6.3 mm. In this example, the seamless capsules could be successfully formed. The powder component in the whole seamless capsule was 10% by weight.

Table 1 mentioned below shows the particle size of the seamless capsule, the ratio of the content to the whole capsule (content ratio:wt %), the ratio of the outer shell to the whole capsule (outer shell ratio:wt %), the ratio of the film to the whole capsule (film ratio:wt %), the content prescription and its specific gravity, % by weight of the bifidobacterium powder in the content prescription, the outer shell prescription and its specific gravity, % by weight of titanium dioxide in the outer shell prescription, the film prescription and its specific gravity, the difference (Δd=d_{B}−d_{A}) between the specific gravity of the outer shell and the specific gravity of the content, % by weight of the powder component in the whole seamless capsule, and suitability of seamless encapsulation.

The suitability of encapsulation in Table 1 was expressed by the following criteria.

o: There was no uneven thickness or the like, and concentric sphere encapsulation could be correctly achieved.

Δ: Although there was a slight misalignment at the center, capsules could be manufactured normally.

x: A degree of uneven thickness was high, or encapsulation was difficult, so that capsules could not be manufactured normally.

### Comparative Example 1

In Comparative Example 1, a seamless capsule was manufactured in the same manner as in Reference Example mentioned above, except that the amount of the bifidobacterium powder was increased to 40% by weight in the content. The specific gravity (d_{A}) of the content increased to 1.068. The difference in specific gravity (d_{B}−d_{A}) between the outer shell and the content was −0.164. In Comparative Example 1, it was difficult to form a seamless capsule. The powder component in the whole seamless capsule was 25.2% by weight.

Similarly to Reference Example, Table 1 shows the particle size of the seamless capsule, the ratio of the content to the whole capsule (content ratio:wt %), the ratio of the outer shell to the whole capsule (outer shell ratio:wt %), the ratio of the film to the whole capsule (film ratio:wt %), the content prescription and its specific gravity, % by weight of the bifidobacterium powder in the content prescription, the outer shell prescription and its specific gravity, % by weight of titanium dioxide in the outer shell prescription, the film prescription and its specific gravity, the difference (Δd=d_{B}−d_{A}) between the specific gravity of the outer shell and the specific gravity of the content, % by weight of the powder component in the whole seamless capsule, and suitability of seamless encapsulation.

### Example 1

In Example 1, although the amount of the bifidobacterium powder was 40% by weight in the content as in Comparative Example 1, the specific gravity (d_{B}) of the outer shell was increased to 1.052 by adding 18% by weight of titanium dioxide into the outer shell prescription. The specific gravity (d_{A}) of the content was 1.068 as in Comparative Example 1. The difference in specific gravity (d_{B}−d_{A}) between the outer shell and the content was −0.016. In Example 1, a seamless capsule could be manufactured normally. The powder component in the whole seamless capsule was 25.2% by weight.

Similarly to Reference Example, Table 1 shows the particle size of the seamless capsule, the ratio of the content to the whole capsule (content ratio:wt %), the ratio of the outer shell to the whole capsule (outer shell ratio:wt %), the ratio of the film to the whole capsule (film ratio:wt %), the content prescription and its specific gravity, % by weight of the bifidobacterium powder in the content prescription, the outer shell prescription and its specific gravity, % by weight of titanium dioxide in the outer shell prescription, the film prescription and its specific gravity, the difference (Δd=d_{B}−d_{A}) between the specific gravity of the outer shell and the specific gravity of the content, % by weight of the powder component in the whole seamless capsule, and suitability of seamless encapsulation.

An enlarged photograph of the seamless capsule obtained is shown as FIG. 4.

### Example 2

In Example 2, the amount of the bifidobacterium powder was increased to 50% by weight in the content, and the specific gravity (d_{A}) of the content was set to 1.164. In addition, 25.6% by weight of titanium dioxide was added into the outer shell prescription, and the specific gravity (d_{A}) of the outer shell was adjusted to 1.138. The difference in specific gravity (d_{B}−d_{A}) between the outer shell and the content was −0.026. In Example 2, a seamless capsule could be manufactured normally. The powder component in the whole seamless capsule was 31.5% by weight.

Similarly to Reference Example, Table 1 shows the particle size of the seamless capsule, the ratio of the content to the whole capsule (content ratio:wt %), the ratio of the outer shell to the whole capsule (outer shell ratio:wt %), the ratio of the film to the whole capsule (film ratio:wt %), the content prescription and its specific gravity, % by weight of the bifidobacterium powder in the content prescription, the outer shell prescription and its specific gravity, % by weight of titanium dioxide in the outer shell prescription, the film prescription and its specific gravity, the difference (Δd=d_{B}−d_{A}) between the specific gravity of the outer shell and the specific gravity of the content, % by weight of the powder component in the whole seamless capsule, and suitability of seamless encapsulation.

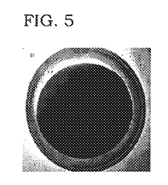

An enlarged photograph of the seamless capsule obtained is shown as FIG. 5.

### Example 3

In Example 3, the amount of the bifidobacterium powder was increased to 60% by weight in the content, and the specific gravity (d_{A}) of the content was set to 1.236. In addition, 25.6% by weight of titanium dioxide was added into the outer shell prescription, and the specific gravity (d_{B}) of the outer shell was adjusted to 1.138. The difference in specific gravity (d_{B}−d_{A}) between the outer shell and the content was −0.098. In Example 3, although there was a slight misalignment at the center, a seamless capsule could be manufactured normally. The powder component in the whole seamless capsule was 37.8% by weight.

Similarly to Reference Example, Table 1 shows the particle size of the seamless capsule, the ratio of the content to the whole capsule (content ratio:wt %), the ratio of the outer shell to the whole capsule (outer shell ratio:wt %), the ratio of the film to the whole capsule (film ratio:wt %), the content prescription and its specific gravity, % by weight of the bifidobacterium powder in the content prescription, the outer shell prescription and its specific gravity, % by weight of titanium dioxide in the outer shell prescription, the film prescription and its specific gravity, the difference (Δd=d_{B}−d_{A}) between the specific gravity of the outer shell and the specific gravity of the content, % by weight of the powder component in the whole seamless capsule, and suitability of seamless encapsulation.

### Example 4

In Example 4, the amount of the bifidobacterium powder was 60% by weight in the content, and the specific gravity (d_{A}) of the content was set to 1.236. In addition, 30.0% by weight of titanium dioxide was added into the outer shell prescription, and the specific gravity (d_{B}) of the outer shell was adjusted to 1.201. The difference in specific gravity (d_{B}−d_{A}) between the outer shell and the content was −0.035. In Example 4, although there was a slight misalignment at the center, a seamless capsule could be manufactured normally. The powder component in the whole seamless capsule was 37.8% by weight.

Similarly to Reference Example, Table 1 shows the particle size of the seamless capsule, the ratio of the content to the whole capsule (content ratio:wt %), the ratio of the outer shell to the whole capsule (outer shell ratio:wt %), the ratio of the film to the whole capsule (film ratio:wt %), the content prescription and its specific gravity, % by weight of the bifidobacterium powder in the content prescription, the outer shell prescription and its specific gravity, % by weight of titanium dioxide in the outer shell prescription, the film prescription and its specific gravity, the difference (Δd=d_{B}−d_{A}) between the specific gravity of the outer shell and the specific gravity of the content, % by weight of the powder component in the whole seamless capsule, and suitability of seamless encapsulation.

### Example 5

Example 5 is an example of a two-layer seamless capsule, and the outer shell corresponds to a film-forming agent layer. In Example 5, the amount of the bifidobacterium powder was 40% by weight in the content, and the specific gravity (d_{A}) of the content was set to 1.068. In addition, the outer shell (referred to as the “film” in Example 5) adjacent to the content was a water-soluble film-forming agent layer containing gelatin, which was used as the film in Examples 1 to 4, and 2.5% by weight of titanium dioxide was added into a film liquid. The specific gravity (d_{B}) was 1.076, and the difference in specific gravity (Δd=d_{B}−d_{A}) between them was 0.008. In Example 5, a seamless capsule could be manufactured normally. The powder component in the whole seamless capsule was 30.8% by weight.

Similarly to Reference Example, Table 1 shows the particle size of the seamless capsule, the ratio of the content to the whole capsule (content ratio:wt %), the ratio of the outer shell to the whole capsule (outer shell ratio:wt %), the ratio of the film to the whole capsule (film ratio:wt %), the content prescription and its specific gravity, % by weight of the bifidobacterium powder in the content prescription, the film prescription and its specific gravity, the difference in specific gravity (d_{B}−d_{A}) between the film and the content, % by weight of the powder component in the whole seamless capsule, and suitability of encapsulation.

### Example 6

In Example 6, a content liquid was prepared by dispersing acetaminophen (average particle size being 48 μm) of Japanese pharmacopoeia in polyethylene glycol 400 (PEG 400) which is a hydrophilic content liquid component such that 40% by weight was achieved. The specific gravity (d_{A}) of the content liquid was 1.159. In addition, 25.6% by weight of titanium dioxide was added into the outer shell prescription, and the specific gravity (ds) of the outer shell was adjusted to 1.138. The difference in specific gravity (d_{B}−d_{A}) between the outer shell and the content was −0.021. In Example 6, a seamless capsule could be manufactured normally. The powder component in the whole seamless capsule was 25.2% by weight.

Similarly to Reference Example, Table 1 shows the particle size of the seamless capsule, the ratio of the content to the whole capsule (content ratio:wt %), the ratio of the outer shell to the whole capsule (outer shell ratio:wt %), the ratio of the film to the whole capsule (film ratio:wt %), the content prescription and its specific gravity, % by weight of the bifidobacterium powder in the content prescription, the outer shell prescription and its specific gravity, % by weight of titanium dioxide in the outer shell prescription, the film prescription and its specific gravity, the difference (Δd=d_{B}−d_{A}) between the specific gravity of the outer shell and the specific gravity of the content, % by weight of the powder component in the whole seamless capsule, and suitability of seamless encapsulation.

### Example 7

Example 7 is an example of a two-layer synthetic polymer film seamless capsule, and the outer shell corresponds to a film-forming agent layer as in Example 5. In Example 7, a content liquid was prepared by dispersing yeast (dry yeast) in MCT (medium-chain fatty acid triglyceride) as a purified product of coconut oil such that 40% by weight was achieved. The specific gravity (d_{A}) of the content liquid was 1.076. In addition, the outer shell (referred to as the “film” in Example 7) adjacent to the content was an aqueous synthetic polymer film-forming agent layer formed of ENTG-3800 (available from Kansai Paint Co., Ltd.), which was a resin compound comprising a polyethylene glycol or a polyethylene glycol skeleton, of which each terminal having a photocurable unsaturated groups. Its specific gravity (ds) was 1.093, and the difference in specific gravity (Δd=d_{B}−d_{A}) between them was 0.017. In Example 7, a seamless capsule could be manufactured normally. The powder component in the whole seamless capsule was 30.8% by weight.

Similarly to Reference Example, Table 1 shows the particle size of the seamless capsule, the ratio of the content to the whole capsule (content ratio:wt %), the ratio of the outer shell to the whole capsule (outer shell ratio:wt %), the ratio of the film to the whole capsule (film ratio:wt %), the content prescription and its specific gravity, % by weight of a dry yeast powder in the content prescription, the film prescription and its specific gravity, the difference in specific gravity (d_{B}−d_{A}) between the film and the content, % by weight of the powder component in the whole seamless capsule, and suitability of encapsulation.

TABLE 1

| | | Reference | Comparative | | | |
|---|---|---|---|---|---|---|
| | | Example | Example 1 | Example 1 | Example 2 | Example 3 |
| Outline of | Particle size (mm) | 6.3 | 6.3 | 6.3 | 6.3 | 6.3 |
| capsule | Content ratio (wt %) | 50 | 63 | 63 | 63 | 63 |
| | Outer shell ratio (wt %) | 27 | 14 | 14 | 14 | 14 |
| | Film ratio (wt %) | 23 | 23 | 23 | 23 | 23 |
| Content (Note 1) | Amount (wt %) of bifidobacterium powder | 20 | 40 | 40 | 50 | 60 |
| | Hardened oil (wt %) | 80 | 60 | 60 | 50 | 40 |
| | Amount (wt %) of acetaminophen | | | | | |
| | Polyethylene glycol 400 (wt %) | | | | | |
| | Dry yeast (wt %) | | | | | |
| | Medium-chain fatty acid triglyceride (MCT) (wt %) | | | | | |
| | Specific gravity (d_{A}) of content | 0.978 | 1.068 | 1.068 | 1.164 | 1.236 |
| Outer | Hardened oil (wt %) | 100 | 100 | 82 | 74.4 | 74.4 |
| shell | Amount (wt %) of titanium dioxide (specific gravity | 0 | 0 | 18 | 25.6 | 25.6 |
| | regulator) | | | | | |
| | Specific gravity (d_{B}) of outer shell | 0.904 | 0.904 | 1.052 | 1.138 | 1.138 |
| Film (Note 2) | Gelatin (wt %) | 18.5 | 18.5 | 18.5 | 18.5 | 18.5 |
| | Glycerin (wt %) | 6 | 6 | 6 | 6 | 6 |
| | Pectin (wt %) | 0.5 | 0.5 | 0.5 | 0.5 | 0.5 |
| | Amount (wt %) of titanium dioxide (specific gravity | | | | | |
| | regulator) | | | | | |
| | Photocurable synthetic polymer (ENTG 3800) (wt %) | | | | | |
| | Polymerization initiator (wt %) | | | | | |
| | Purified water (wt %) | 75 | 75 | 75 | 75 | 75 |
| | Specific gravity of film | 1.055 | 1.055 | 1.055 | 1.055 | 1.055 |

| | | | | | |
|---|---|---|---|---|---|
| Difference in specific gravity (d_{B} − d_{A}) between outer shell and | −0.074 | −0.164 | −0.016 | −0.026 | −0.098 |
| content | | | | | |
| Amount (wt %) of powder component in the whole seamless | 10 | 25.2 | 25.2 | 31.5 | 37.8 |
| capsule | | | | | |
| Suitability of seamless encapsulation | ◯ | X | ◯ | ◯ | Δ |

| | | Example 4 | Example 5 | Example 6 | Example 7 |
|---|---|---|---|---|---|
| Outline of | Particle size (mm) | 6.3 | 6.3 | 6.3 | 6.3 |
| capsule | Content ratio (wt %) | 63 | 77 | 63 | 77 |
| | Outer shell ratio (wt %) | 14 | 0 | 14 | 0 |
| | Film ratio (wt %) | 23 | 23 | 23 | 23 |
| Content (Note 1) | Amount (wt %) of bifidobacterium powder | 60 | 40 | | |
| | Hardened oil (wt %) | 40 | 60 | | |
| | Amount (wt %) of acetaminophen | | | 40 | |
| | Polyethylene glycol 400 (wt %) | | | 60 | |
| | Dry yeast (wt %) | | | | 40 |
| | Medium-chain fatty acid triglyceride (MCT) (wt %) | | | | 60 |
| | Specific gravity (d_{A}) of content | 1.236 | 1.068 | 1.159 | 1.076 |
| Outer | Hardened oil (wt %) | 70 | | 74.4 | |
| shell | Amount (wt %) of titanium dioxide (specific gravity | 30 | | 25.6 | |
| | regulator) | | | | |
| | Specific gravity (d_{B}) of outer shell | 1.201 | | 1.138 | |
| Film (Note 2) | Gelatin (wt %) | 18.5 | 16 | 18.5 | |
| | Glycerin (wt %) | 6 | 6 | 6 | |
| | Pectin (wt %) | 0.5 | 0.5 | 0.5 | |
| | Amount (wt %) of titanium dioxide (specific gravity | | 2.5 | | 2 |
| | regulator) | | | | |
| | Photocurable synthetic polymer (ENTG 3800) (wt %) | | | | 37 |
| | Polymerization initiator (wt %) | | | | 1 |
| | Purified water (wt %) | 75 | 75 | 75 | 60 |
| | Specific gravity of film | 1.055 | d_{B} = 1.076 | 1.055 | d_{B} = 1.093 |

| | | | | |
|---|---|---|---|---|
| Difference in specific gravity (d_{B} − d_{A}) between outer shell and | −0.035 | 0.008 | −0.021 | 0.017 |
| content | | | | |
| Amount (wt %) of powder component in the whole seamless | 37.8 | 30.8 | 25.2 | 30.8 |
| capsule | | | | |
| Suitability of seamless encapsulation | Δ | ◯ | ◯ | ◯ |

All specific gravities are values at 50° C.
Note 1: Suspension containing powder component
Note 2: In Examples 5 and 7, since there are two layers, the film corresponds to the outer shell recited in Claims, and its specific gravity is represented by d_{B}.

As apparent from the above examples, comparative examples, and reference examples, when the specific gravity of the content is 1 or more (d_{A}≥1) and the difference in specific gravity between the content and the outer shell (the film adjacent to the content in Examples 5 and 7) adjacent to the content is within the range of −0.15 to +0.05 (Δd=d_{B}−d_{A}=−0.15 to +0.05), the seamless capsule is suitably manufactured. Although the reference example is an example of a bifidobacterium powder-containing seamless capsule at the present time, in this example the specific gravity of the content is less than 1, and a seamless capsule can be manufactured with a usual outer shell and film prescription. Although Comparative Example 1 is an example in which the specific gravity of the content is 1 or more, in this example the difference (Δd=d_{B}−d_{A}) between the specific gravity of the content and the specific gravity of the outer shell is −0.164, so that seamless encapsulation is difficult. According to the present invention, it can be seen that although the content of the powder component in the whole seamless capsule is 10% by weight in the reference example, in Examples all seamless capsules can contain 25% or more by weight of the powder component.

### INDUSTRIAL APPLICABILITY

The present invention provides a seamless capsule capable of containing a large amount of a powder component in its content. A seamless capsule obtained by this invention can contain a large amount of a bifidobacterium powder, other beneficial enterobacterium, drugs, and the like, to thereby expect effects such as improvement in QOL (quality of life) by reduction in the number of capsules to be taken, reduction in manufacturing cost, and effective use of resources. In addition, even in bioreactor applications where yeast and useful bacteria are seamlessly encapsulated with a synthetic polymer membrane, a large amount of yeast and bacteria powder can be contained in the capsule, so that it is expected to improve efficiency of the manufacturing process by, for example, shortening reaction time and miniaturizing a reaction apparatus.

### REFERENCE SIGNS LIST

A: Nozzle section
B: Seamless capsule jet
1: Inner nozzle
2: Intermediate nozzle
3: Outer nozzle
4: Capsule content liquid
5: Outer shell liquid
6: Outermost layer liquid

1-15. (canceled)

16. A powder component-containing seamless capsule comprising:
a content; and
an outer shell enclosing the content adjacently to the content,
wherein the content is a suspension prepared by dispersing a powder component that is poorly soluble in water and oil, in an oil component or a hydrophilic component, the content has a specific gravity of 1 or more, and a difference (Δd=d_{B}−d_{A}) between the specific gravity (d_{A}) of the content and a specific gravity (d_{B}) of the outer shell is within a range of −0.15 to +0.05.

17. The powder component-containing seamless capsule according to claim 16, wherein one or more outermost layers enclosing the outer shell are provided on an outer side of the outer shell.

18. The powder component-containing seamless capsule according to claim 16, wherein the specific gravity of the outer shell is adjusted by adding a specific gravity regulator.

19. The powder component-containing seamless capsule according to claim 18, wherein the specific gravity regulator is selected from the group consisting of inorganic or organic powder having a particle density of 0.9 to 6.0 g/cm³ and a mixture thereof.

20. The powder component-containing seamless capsule according to claim 16, wherein the content has a specific gravity of 1.0 to 1.4.

21. The powder component-containing seamless capsule according to claim 16, wherein the powder component has an average particle size of more than 20 μm and 150 μm or less.

22. The powder component-containing seamless capsule according to claim 16, wherein the powder component is a beneficial enterobacterium.

23. The powder component-containing seamless capsule according to claim 16, wherein the content is a suspension prepared by dispersing a bifidobacterium powder in the oil component.

24. The powder component-containing seamless capsule according to claim 16, wherein the outer shell is a hardened oil whose specific gravity is adjusted to 1.0 to 1.4 by the specific gravity regulator.

25. The powder component-containing seamless capsule according to claim 17, wherein the outermost layer is a monolayer and comprises a water-soluble film-forming agent.

26. The powder component-containing seamless capsule according to claim 16, wherein the powder component is contained in the content in an amount of 20 to 60% by weight, based on a weight of the content.

27. A three-layer bifidobacterium powder-containing seamless capsule comprising:
a content in which a bifidobacterium powder is dispersed in an oil component and that has a specific gravity of 1.0 to 1.4;
an outer shell that is a hardened oil whose specific gravity is adjusted to 1.0 to 1.4 by a specific gravity regulator and encloses the content adjacently to the content; and
an outermost layer that is a monolayer and comprises a water-soluble film-forming agent,
wherein a difference (Δd=d_{B}−d_{A}) between the specific gravity (d_{A}) of the content and the specific gravity (d_{B}) of the outer shell is within the range of −0.15 to +0.05.

28. A method for manufacturing the powder component-containing seamless capsule according to claim 16, comprising simultaneously extruding a content liquid from an inner nozzle of a double nozzle having a sequentially increasing radius and disposed concentrically and an outer shell liquid from an outer nozzle into a cooling liquid,
wherein the content liquid is a suspension prepared by dispersing a powder component that is poorly soluble in water and oil, in an oil component or a hydrophilic component, the content liquid has a specific gravity of 1 or more, and a difference (Δd=d_{B}−d_{A}) between the specific gravity (d_{A}) of the content liquid and a specific gravity (d_{B}) of the outer shell liquid is controlled within the range of −0.15 to +0.05.

29. The method for manufacturing a powder component-containing seamless capsule according to claim 28, wherein one or more outermost layer nozzles having a sequentially increasing radius and arranged concentrically are further provided outside the double nozzle, and one or more outermost layer liquids are extruded from the one or more outermost layer nozzles into the cooling liquid simultaneously with the content liquid and the outer shell liquid.

30. A method for manufacturing the three-layer bifidobacterium powder-containing seamless capsule according to claim 27, the method comprising simultaneously extruding a content liquid from an inner nozzle of a triple nozzle having a sequentially increasing radius and comprising the inner nozzle, an intermediate nozzle, and an outer nozzle arranged concentrically, an outer shell liquid from the intermediate nozzle, and an outermost layer liquid from the outer nozzle into a cooling liquid,
wherein the content liquid is a suspension prepared by dispersing a bifidobacterium powder in the oil component and has a specific gravity of 1.0 to 1.4, the outer shell liquid is a hardened oil whose specific gravity is adjusted to 1.0 to 1.4 by the specific gravity regulator, the outermost layer liquid contains the water-soluble film-forming agent, and a difference (Δd=d_{B}−d_{A}) between the specific gravity (d_{A}) of the content liquid and a specific gravity (d_{B}) of the outer shell is controlled within the range of −0.15 to +0.05.
